(19) United States
(12) Patent Application Publication
(10) Pub. No.: US 2007/0151565 A1
(43) Pub. Date: Jul. 5, 2007

(54) Method for the production and positioning of an intrauterine device which is adapted to the uterine cavity measurements

(76) Inventor: Guillermo Sanders Acedo, Mexico DF (MX)

Correspondence Address: DEFILLO & ASSOCIATES, INC., P.O. Box 14104, Clearwater, FL, 33766, US

(21) Appl. No.: 10/580,328

(22) PCT Filed: Sep. 22, 2004
(86) PCT No.: PCT/MX04/00068

§ 371 Date: May 24, 2006

(30) Foreign Application Priority Data
Oct. 16, 2003 (MX) PA/A/2003/009458

(51) Int. Cl. A61F 6/14; A61B 5/103; A61B 5/117

(52) U.S. Cl. 128/839; 600/591

(57) ABSTRACT

The invention relates to a method of producing an adaptable or made-to-measure intrauterine device. The inventive method consists in: determining physiological variables of the uterine cavity of the patient; producing the device in accordance with the patient's measured physiological variables; and positioning the device using an insertion element that has been specially designed for said purpose. The invention also relates to the intrauterine device which can be adapted to the individual measurements of the uterine cavity of a patient and which is produced using the aforementioned method.

### BACKGROUND

In spite of efforts by humanity since ancient times to control fertility, it was not until the middle of the past century that recognized scientific advancements were carried out which took shape with the appearance of synthetic hormones and almost simultaneously with modern intrauterine devices as a dependable alternative to control said human fertility. Since then three generations of devices have come out to control the fertility of couples: the first of these consisted of products manufactured with metallic and organic material, the Graefner Ring, which incorporated alloys of copper, nickel and zinc; in 1934, the Ota Ring, etc. The second generation came out in the 60's with the Margullies Coil made of plastic material with barium sulfate as a radiopaque barrier, and the double S of D. Jack Lippes which had four models, each one with different dimensions. Later was the appearance of other shapes and models containing cylinders or a copper filament in the most diverse presentations up to the most recent appearance of intrauterine devices releasing synthetic human hormones into the uterine cavity.

Since then, although several decades have passed since the appearance of intrauterine devices, the industry favored, with some exceptions, the standardization of a contraceptive in the shape of a T and a filament and cylinder wound with copper, which has a world-wide acceptance.

In spite of its widespread circulation, many users from the beginning stated their rejection of the standardization of said device, and stating constant complaints which caused the removal of the device from the market. These complaints consisted of pains, cramps, profuse bleeding, colic and discharges which finally lead to the stopping of the use thereby reaching between 30 and 50%. See Praxis Dr. Med. Eric Miller, “Contraceptive Family Planning”, 20 Jan., 2003.

As a Consequence, on comparing the original models with the products presently available, we find inexplicable and unacceptable differences of 32% on the horizontal piece and 22.5% on the vertical shaft piece. In this way, placing a device whose horizontal piece is up to 32% longer than the width of the uterus of a woman who has never pregnant, is a demonstration of a lack of interest, which has prevailed throughout the years, by the suffering endured by the young people (adolescents), and represents a social tragedy because of the hundreds of thousands of abortions carried out and unwanted births occurring. In this way, the principal objective of the present invention is to solve the problem of the comparability between the uterine cavity and a device which adapts its dimensions to the measurements of the uterus in accordance with this development during the process of maturing, as well as the change of the measurements produced from childbirths.

The objective of the present invention is the manufacturing of a made-to-measure intrauterine device of the uterine cavity.

### ABSTRACT OF THE INVENTION

In accordance with the present invention, a system of manufacturing an intrauterine device conforming to the personal measurements of each user is described, which consists of:
determining various physiological variables of the uterine cavity of every particular patient,
manufacture the device in accordance with the individual measurements of the patient,
place the device with the apparatus specially designed for this purpose.

Among the physiological variables to determine are the dimensions of the uterine cavity of each particular patient.

Equally described is an intrauterine device adapted to the particular measurements of the uterine cavity of a patient obtained by said method.

Moreover, in some modalities, you can use energy fields to achieve the anatomical representation and desired measurements. So, in a particular modality of the method of this invention a device equipped with a sensor is supplied which is inserted in the body of a patient and one or more apparatuses for the creation of a field of energy (for example one or several magnets), one or several ultrasonic generators, one or several light sources , one or several radiofrequency generators, one or several x-ray element contrasts, one or several sources of infra-red, one or several sources of microcurrents, etc, which can be used to create electromagnetic fields or another field of energy( that is, fields of detection) around and/or inside the body of the patient. One means which supplies signals and supervision of the sensor (for example a computer/controller and monitor)is later utilized to receive signals from it or the sendors and to provide, on the basis of these signals, markers or signals of the placement and/or orientation of each sensor within the field of detection. In some applications, the specific anatomy of all or a part of the uterine cavity can be represented utilizing techniques known as hysterosalpingography image formation.

Additional objectives, aspects and elements of this invention will be apparent for those skilled in the specialty of reading and comprehending the detailed description and the claims established and continuing with the accompanied drawings and/or attached documents.

### BRIEF DESCRIPTION OF THE SYSTEM

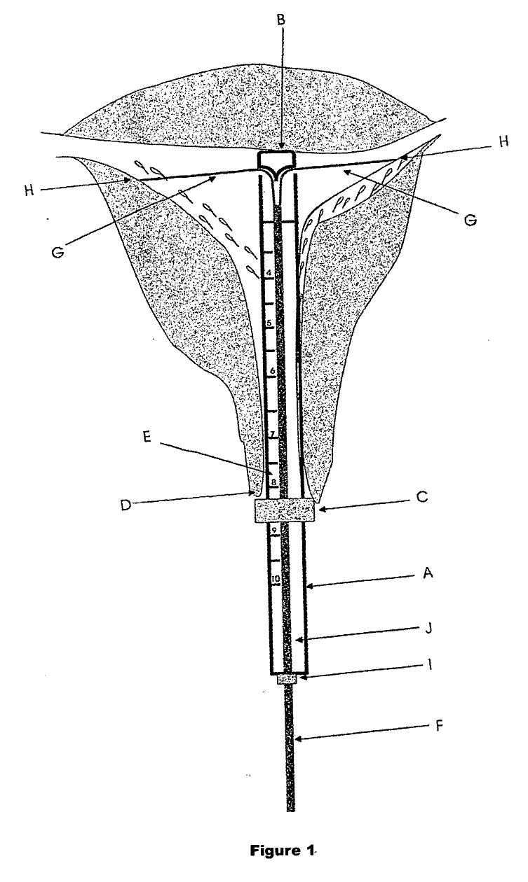

The effectiveness of the Intrauterine Device depends, to a large extent, on its adaption to the dimensions of the feminine uterine cavity , therefore the procedure is started with the complete and precise measurement of the uterine cavity through the utilization of a double function hysterometer: FIG. 1.

Double-Function Hysterometer (FIG. 1): It is an apparatus which permits measuring the dimensions of the uterine cavity, its length as well as its width for the purpose of being able to prescribe the size of the contraceptive intrauterine device appropriate for each woman.

Once the vaginal speculum is employed and the vagina is made aseptic, the length of the uterine cavity is measured introducing the tube or the principal measuring unit of the cylinder touching the back of the cavity of the apparatus (A) through the orifice of the neck of the uterus until touching the back of the cavity, where a light resistance is felt (B). Once this action has been done, the cervical extreme is slid until touching the neck of the uterus (D) and one can observe on the scale engraved on the tube, in centimeters, the depth of the cavity.

The width of the uterine cavity is measured introducing the width-measuring plunger of the uterine cavity (F) inside the principal tube until touching the back of the principal cylinder, being careful that the two measuring devices (G) remain in a horizontal position, one to the right and the other to the left, and using greater pressure, the measuring device extremes move towards the side until touching the lateral walls of the uterine cavity, until where a light resistance is noticed (H). At this point the measuring device extreme end (I) is slid over the width measurement device scale (J), in millimeters, and on removing this plunger you can read the width of the uterine cavity on the scale.

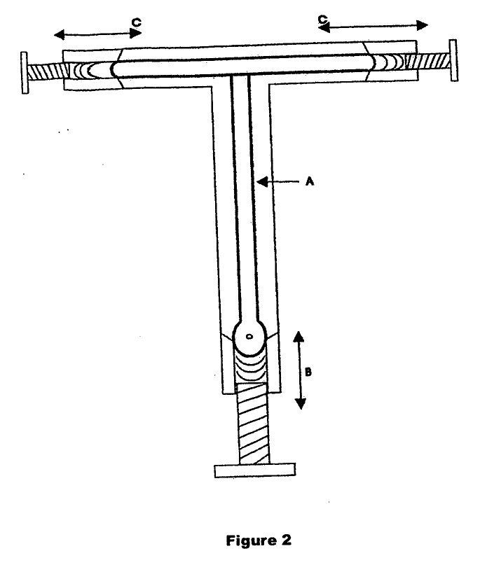

Once the individual measurements of the uterine cavity are recorded (E & J of FIG. 1), they are translated to the mold (FIG. 2)which is placed in the plastic injector to mold the piece and for illustrative purposes a “T shape” is used whose size and shape can be changed.

The invention being registered, as previously mentioned, consists of a system for the manufacturing of an intrauterine device measured to the uterine cavity of each user and in accordance with these, the manufacturing of a frame that can have diverse shapes on which is placed a cylindrical filament or a copper sleeve with 380 square millimeters of medical grade copper. This quantity can vary depending on the model of the device.

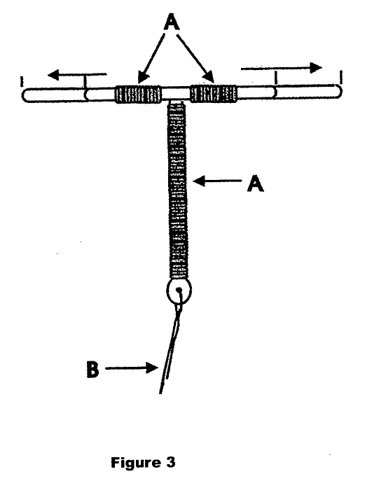

For the best illustrative effect of the method described the example utilized is a frame in the shape of a “T” (A) which increases or reduces, according to each case 0.1 millimeters along its vertical length (B) and at the same time 0.1 millimeters along its horizontal part (C). Around the frame is wound a copper filament (A FIG. 3) on the horizontal arm as well as the vertical arm for greater contraceptive protection. However the said invention is not limited to a determined form, so it can, without reducing its efficiency, modify or adapt itself in accordance with the personal anatomical necessities recommended in the medical practice of gynecology.

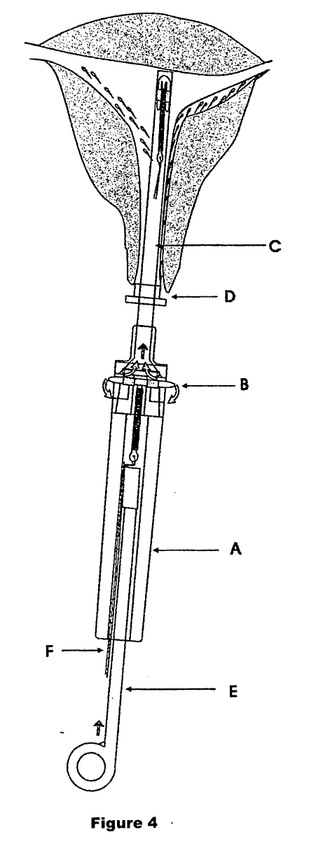

Once the piece is molded (FIG. 3), a plastic thread is incorporated in the device (B), which can be incorporated through a mold or tied. The unit is sterilized and is placed on the Introducer apparatus of the intrauterine device (FIG. 4 ). This apparatus or system, designed to facilitate the introduction of the copper “T” (IUD) of the intrauterine device, consists of a support sleeve (A) in whose distal end is placed the IUD (B) with the appropriate measurement (In accordance with the measurements of the uterine cavity, introducing its shaft into the interior of the tube, watching that the monofilamet threads (F) remain free to one side. To this sleeve the tube inserter (C) is mounted and with the vaginal speculum already placed , the “NO TOUCH” technique is introduced through the orifice of the neck of the uterus to the back of the cavity, fixing the arms of the horizontal part of the IUD in a horizontal position, one arm to the right side and the other to the left. Once it is done, the inserter tube is removed approximately 1 centimeter and the cervical limit (D) is slid until touching the cervix. Now the applicator plunger is introduced (E) in the sleeve and applying pressure the IUD is pushed through the inserter tube (At this moment the arms of the horizontal part of the IUD are folded in order to pass through the interior of the inserter tube) until reaching the depth of the uterine cavity where, thanks to its own plastic elasticity memory of the IUD, the arms of the IUD are unfolded remaining in the correct position in the back of the uterus. To check its correct placement holding the inserter tube in its position (C), the cervical limit (D) is slid back and the inserter tube is introduced again to the back of the uterus. Once this step is done the sleeve (A) and the inserter tube are removed being careful not to pull the monofilament threads of the IUD, in order to avoid its moving, and once these are pulled back the threads are cut from one to two centimeters from the neck of the uterus. Once these steps are done you can remove the vaginal speculum.

Another aspect of the invention includes methods to represent physiological variables inside the body of the patient. A method in accordance with this aspect of the invention includes the steps to detect one or more physiological variables and to determine the morphological conformation of the uterine cavity and in this way to provide a measurement associated with the position and form. More preferably, methods in accordance with this aspect of the invention include the additional steps to repeat the previously mentioned steps to provide a variety of measurements associated with a diversity of positions, and in this way to provide a map in the shape of the uterine cavity. The method can furthermore include the step to show this representation or map like a visible image such as a connection of lines making an outline, areas of different color or areas of different contrast, with or without other characteristics derived from other modalities of image formation.

The method as well as determining physiological variables of the uterine cavity of the particular patient, includes the manufacturing of the device in accordance with said individual characteristics and measurements of the patient. In one modality, the physiological variables to be determined consists of the dimensions of the uterine cavity of a particular patient measured with a hysterometer, which can be made of disposable material.

The invention also refers to an intrauterine device adaptable to the particular measurements of the uterine cavity of a patient which is obtained by the method of the said invention in its diverse modalities.

The device described here should be judged with reference to the actual modalities of the said invention, but more diverse additions, alterations and modifications to those modalities can be carried out without getting away from the nature and reach expected of the invention. All of these reasonable additions, modifications and alterations will have to be considered equivalents of the modalities described and must be included within the scope of the following claims.

### BIBLIOGRAPHY

1. Population Reports, “The IUD's Today, December 1995, Volume XXIII, Number 5 Series B, Number 6.
2. Trussel Et Al “Studies in Family Planning”. 21511990
3. SA DGSML “The adolescent woman, adult woman, an old woman and their health” Mexico 1992
4. “Studies carried out by Croxatto, H. B., Moyer, d. L. Tredway, D. R. and Ortiz, M. D. mentioned in “Technical Report Series No 753 from World Health Organization, Geneva, 1987.
5. “The Economic Value of Contraception: A Comparison of 15 Methods”, James Trussell, PhD, Joseph A Leveque, MD, MBA, Jacqueline D. Koenig, Robert London, MD, Spencer Borden, MD, MBA, Joan Hennebery, Katherine D. LaGuardia, MD, MPH, Felicia Stewart, MD, T. George Wilson, MD, PhD, Susan Wysocki, RNC, NP, and Michael Strauss, MD, MPH. American Journal of Public Health April 1995 Vol. 85 No. 4
6. James Trussell, Jacqueline Koenig, Felicia Stewart, and Jacqueline E. Dorroch, “Medical Care Cost Savings From Adolescent Contraceptive Use”, Family Planning Perspectives Vol. 29 No. 6, November/December 1997.
7. David Hubacher, Matthew Holtman, Miriam Fuentes, Gregorio Perez-Palacios, Barbara Janowitz, “increasing efficiency to meet future demand: Family planning services provided by the Mexican Ministry of Health”, International Family Planning Perspectives, Vol. 25, No. 3 September 1999;119-1244 and 138.
8. Report prepared by Katherine Treiman, M. P., Laurie Sc, Adrienne Kois M A and Ward Rinehart M. A. This number is an update of Popular Reports, “The IUDs—A New Examination” Series B No. 5, December 1995.
9. Tejuja, S. and Malkani, P. K.: Clinical significance of correlation between size of uterine cavity and IUD. A study by planimeter-hysterogram technique. Am J. Obstet. Gynecol. 1 05:620,1969.

1. A method for producing a made-to-measure intrauterine device, characterized in that it consists in: determining physiological variables of the uterine cavity of the particular patient; producing the device in accordance with the physiological variable measurements of the patient.

2. A method in accordance with claim 1, characterized in that the physiological variables to be determined consists in the dimensions of the uterine cavity of the particular patient.

3. A method in accordance with claim 1 or 2 characterized in that the determination of the dimensions of the uterine cavity of the patient are done with a hysterometer which measures the physiological variables of the user, which may or may not be disposable, just as any other that available technology permits for exploitation.

4. A method in accordance with claims 1 to 3, characterized in that it includes the additional steps of repeating the steps in order to provide a number of measurements associated with a diversity of positions, in order to provide in this way a map of the physiological variables over a number of positions.

5. An intrauterine device adaptable to the particular measurements of the uterine cavity of a patient which is obtained by the method in accordance with claims 1 to 4.

6. An apparatus designed especially for the placement of an intrauterine device in the shape of a “T”.
